(19) United States
(12) Patent Application Publication
(10) Pub. No.: US 2009/0068300 A1
(43) Pub. Date: Mar. 12, 2009

(54) PRE- AND POST-WORKOUT FORMULATIONS PROCESSES AND SUPPLEMENT PRODUCTS THEREBY

(75) Inventor: Paul P. Edalat, Santa Ana, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: VERIPAK, INC., SANTA ANA, CA (US)

(21) Appl. No.: 12/205,728

(22) Filed: Sep. 5, 2008

Related U.S. Application Data
(60) Provisional application No. 60/970,679, filed on Sep. 7, 2007.

(51) Int. Cl. A61K 36/899; A61K 31/195; A61P 3/02

(52) U.S. Cl. 424/776; 514/561

(57) ABSTRACT

An improved process for manufacturing, using, and optimizing performance of a mammal using a pre- and post-workout supplement discloses important ingredients, formulations, and dosage protocols. Elite athletes, average consumers, and select groups of those challenged can be benefited by products by the disclosed processes, as evinced by the performance of UFC once and future light-heavyweight Champion of the World, Chuck “The Iceman” Liddell at UFC 88 in Atlanta, Ga. on 6 Sep. 2008.

### CROSS REFERENCE TO RELATED APPLICATIONS

The present application claims full Paris Convention Priority of U.S. Provisional Patent Application 60/970,679 filed Sep. 7, 2007 in the name of the present inventors, and assigned to the instant assignee. Said document is expressly incorporated herein by reference as if fully set forth herein, and full Paris Convention and U.S. Patent Law codified priority benefits expressly claimed with the instant filing.

### BACKGROUND OF THE DISCLOSURE

1. Field of the Disclosure

The present disclosure relates to tablets for use by those engaging in physical exercise. In particular, the present disclosure offers for consideration an improved pre- and post-workout formulation set, especially effective when used for high performance athletic endeavors. This disclosure relates to processes for generating such improved and enhanced formulations, and products by the processes, inter alia.

Both the groups of people who are average citizens and elite athletes often need to engage in serious physical activity, and to bolster and re-stock their bodies' respective reserves of key minerals, vitamins, and precursors to the same. Likewise, select challenged individuals can benefit from use of supplements to be eligible for relatively intense and vigorous physical training using supplements, as shall be elucidated within the context of the instant application and claims appended hereto.

### SUMMARY OF THE DISCLOSURE

Mixed martial arts (“MMA”) has become the sport of choice among large segments of the populace, and is embodied in optimal form in the ULTIMATE FIGHTING CHALLENGE (“UFC”), based upon viewer-ship statistics and the experience level of the involved participants. The present invention provides insight into how elite athletes prepare for, and compete at such high levels of performance.

Referring now to the appendix of U.S. Provisional Patent Application 60/970,679 filed Sep. 7, 2007—in addition to the articles in the information disclosure statement filed contemporaneously herewith, Chuck “The Iceman” Liddell is offered for consideration as a proven champion of MMA, and a spokesperson for the future of supplementation, as described herein, and claimed below.

The event known as UFC 88 features Chuck Liddell, bauded up to full speed with the support of the instant regimens, against a younger and physically more imposing Rashad Evans, on 6 Sep. 2008, in Atlanta, Ga. Those skilled in the art readily understand that the teachings of the present invention shall be empirically validated with the outcome of this event, as described below and claimed herein.

Briefly stated, an improved process for manufacturing, using, and optimizing performance of a mammal using a pre- and post-workout supplement discloses important ingredients, formulations, and dosage protocols. Elite athletes, average consumers, and select groups of those challenged can be benefited by products by the disclosed processes, as evinced by the performance of the UFC once and future Light Heavyweight Champion of the World, Chuck “The Iceman” Liddell at UFC 88 in Atlanta, Ga. on 6 Sep. 2008.

According to embodiments there is disclosed a process for manufacturing an improved pre-workout formulation set which comprises, in combination, at least Tribulus Terrestris Extract Standardized To 40% Furastanol Saponins, D-RIBOSE, Magnesium aspartate, Caffeine, Zinc Methionine, Pyridoxine, Beta-Alanine, Chasetree 4:1 Extract, Avena Savita 10:1 Extract, Epimedium 4:1, and Eurycoma Longolia.

According to embodiments, there is disclosed a process for manufacturing an improved post-workout formulation set, comprising, in combination, at least Creatine, trace minerals, Glutamine, Lysine, Isoleucine, Threonine, Leucine, Netbione and Valine.

According to embodiments, there is disclosed, a process for combining the resultant pre- and post-workout formulation/blend with a plurality of granulated ingredients for an improved supplement package.

According to the teachings of the instant disclosure, processes and products thereby may be formulated into specific daily dosages and weights, and/or customized for particular elite athletes, as known to those of skilled in the art.

According to embodiments of the present invention there is disclosed a process for optimizing athletic performance in mammals, comprising, in combination; providing a pre-workout supplement complex to be ingested by a subject, providing a post-workout supplement complex to be ingested by a subject, and instructing said subject on timing and dosage of each said pre-workout supplement complex to be ingested by a subject and each said post-workout supplement complex to be ingested by a subject.

### DETAILED DESCRIPTION OF THE DISCLOSURE

The present inventors have discovered that a daily dosage or variable pre- and post-workout set can be formulated, capsuled or entabulated or otherwise administered and offered for consideration which is composed of essential athletic performance enhancing or physiologically stabilizing ingredients. As mentioned above, and discussed and claimed below, this progress satisfies a number of longstanding and unmet needs, in offering for consideration an effective supplement, a goal of many and the accomplishment of few to date, given consumer needs, without other downstream issues, such as being illegal, or dangerous to users or subjects.

Expressly incorporated herein by reference are the following U.S. Letters patents, each as if fully set forth herein: U.S. Pat. Nos. 7,247,324; 6,953,588; 6,903,136; 6,814,983;, 6,673,378; 6,299,896; 5,942,255; 5,817,329, each of which has been found to be different from the teachings of the present invention, although helpful in defining the state of the art.

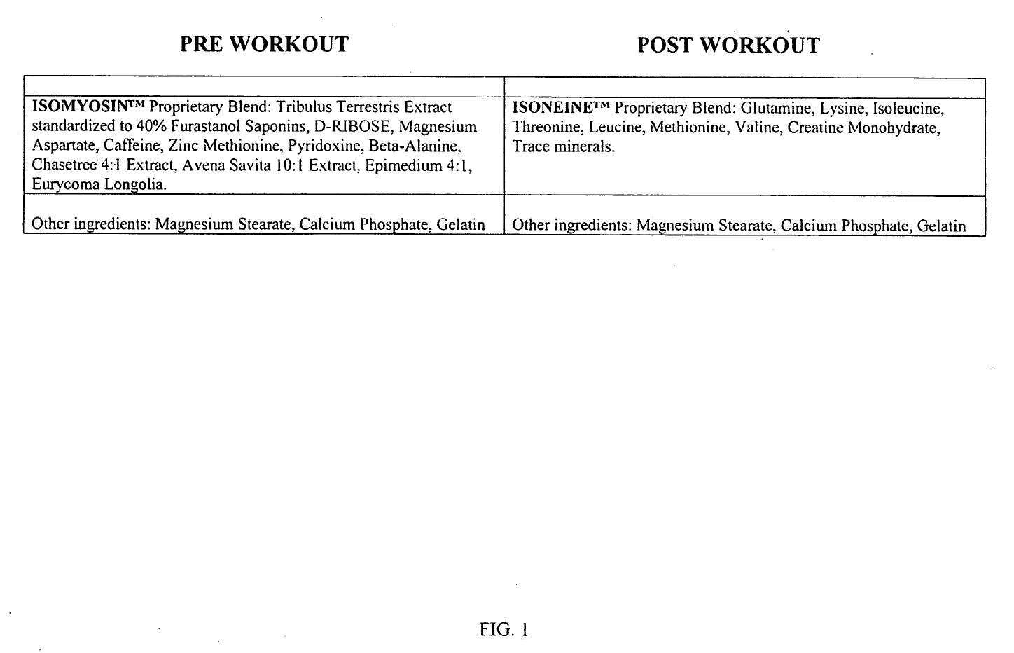

Chuck Liddell, once and future Light Heavyweight Champion of the UFC, is regarded as among the dominant MMA Champions of all time. At age 38, fighting around a weight of 205 lbs., the years spent fine-tuning his game have taught him precisely how important nutrition is to his ability to put his best foot forward. Not unlike Lance Armstrong, who measured output by watts of power generated to maintain dominance of the Tour de France, Chuck Liddell realizes making nutrition a cornerstone is essential for optimizing his physical ability to battle within the Octagon. Being meticulous about the regimens which he follows, and the fuels which his body burns has kept Liddell competitive and at peak physical condition enabling him to sustain massive punishment, while dishing out the same to his competitors. As a part of an elite class of athletes Chuck Liddell uses the formulation, substantially as set forth in FIG. 1, before and after his intense workouts.

For the purposes of this specification, “ISOMYOSIN™” is defined as a proprietary blend of ingredients as set forth in FIG. 1, as is “ISONEINE™” likewise defined according to FIG. 1.

According to embodiments, athletic performance may be optimized by using ISOMYOSIN™, or Tribulus Terrestris Extract standardized to 40% Furastanol Saponins, D-RIBOSE, Magnesium aspartate, Caffeine, Zinc Methionine, Pyridoxine, Beta-Alanine, Chasetree 4:1 Extract, Avena Savita 10:1 Extract, Epimedium 4:1, Eurycoma Longolia pre-workout, and it has been discovered that high performance physiological challenges are mitigated. Likewise, ISONEINE™ or Creatine, trace minerals, Glutamine, Lysine, Isoleucine, Threonine, Leucine, Nethione and Valine for post-workout.

Those skilled in the art are aware that elite athletes often push performance envelopes and require enhanced pre- and post-exercise supplements. Likewise, the instant formulation compounds well without the need for many of the extraneous additives that others resort to. Likewise, testing shows that the resulting effect leverages from the strength of the more traditional ingredients to satisfy the needs of retail customers in parity with known products.

Referring now again to FIG. 1, a typical formulation is offered for consideration, available from Iceman Nutrition LLC and/or VERIPAK, INC. of Santa Ana, Calif. 92705.

Those skilled in the art will readily understand how manufacturing of the instant disclosure proceeds, based on this description, and the claims appended hereto. Namely, after protocols are followed in validating the weight and identity of all materials to be used, “powder/crystalline/granular” ingredients are added. Upon adding of final ingredients proper amounts of time need to be allotted for even consistency of all active ingredients.

While the formulation and methods have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

While the methods and agents have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety, of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant(s).

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A process for manufacturing a pre-workout supplement, which comprises, in combination:
providing at least:
Tribulus Terrestris Extract standardized to 40% Furastanol Saponins, D-RIBOSE, Magnesium aspartate, Caffeine, Zinc Methionine, Pyridoxine, Beta-Alanine, Chasetree 4:1 Extract, Avena Savita 10:1 Extract, Epimedium 4:1, Euiycoma Longolia; and, Magnesium Stearate, Calcium Phosphate and Gelatin a for a pre-workout supplement.

2. A product by the process of claim 1.

3. The product of claim 2, whereby a daily dosage formulation ranges from at least about 1,920 mg to approximately 1,950 mg.

4. A process for manufacturing a post-workout supplement, which comprises, in combination:
providing at least:
Creatine Monohydrate; Glutamine; Lysine; Isoleucine; Threonine; Leucine; Methionine and Valine, Magnesium Stearate, Calcium Phosphate and Gelatin a for a post-workout supplement.

5. A product by the process of claim 4.

6. The product of claim 5, whereby a daily dosage formulation ranges from at least about 1,900 mg to approximately 1,930 mg.

7. A kit, comprising Tribulus Terrestris Extract standardized to 40% Furastanol Saponins, D-RIBOSE, Magnesium aspartate, Caffeine, Zinc Methionine, Pyridoxine, Beta-Alanine, Chasetree 4:1 Extract, Avena Savita 10:1 Extract, Epimedium 4:1, Eurycoma Longolia; and, Magnesium Stearate, Calcium Phosphate and Gelatin a for a pre-workout supplement and Creatine Monohydrate; Glutamine; Lysine; Isoleucine; Threonine; Leucine; Methionine and Valine, Magnesium Stearate, Calcium Phosphate and Gelatin a for a post-workout supplement.

8. The kit defined in claim 7, further comprised of two separate sets of capsules, tablets and packaging for the same.

9. A process for optimizing athletic performance in mammals, comprising, in combination:
providing a pre-workout supplement complex to be ingested by a subject;
providing a post-workout supplement complex to be ingested by a subject; and,
instructing said subject on timing and dosage of each said pre-workout supplement complex to be ingested by a subject and post-workout supplement complex to be ingested by a subject.

10. A product by the process of claim 9.

11. A method of supplementing the diet of a performance athlete, which comprises, in combination:
administering prior to exercise an aliquot equivalent to at least an established daily value of a pre-workout supplement set for testosterone boosting, power and energy, acceleration, thermogenesis, and energy recovery; and,
administering following exercise an aliquot equivalent to at least an established daily value of a post-workout supplement set for anti-catabolism, protein synthesis, cell volumization, growth stimulation and muscle recovery.

12. The method of claim 10, the pre-workout supplement aliquot being ISOMYOSIN™.

13. The method of claim 10, the post-workout supplement aliquot being ISONEINE™.

14. The method of claim 10, said ISOMYOSIN™ aliquot comprising at least about 1,926 mg. of supplement and said ISONEINE™ aliquot comprising at least about 1,910 mg. of supplement.

15. A kit comprising at least a container of ISOMYOSIN™ and ISONEINE™ and directions for use.

16. In a method for optimizing athletic performance output in healthy mammals, the improvements comprising, in combination:
providing a pre-workout supplement formulation allowing a user to endure strenuous exercise while approaching the user's limits of performance;
providing a post-workout supplement formulation allowing a user to replenish required elements and to prepare for future training;
and procuring strength, stamina and recovery gains without the use of illegal or dangerous ingredients.

17. The method of claim 16, further comprising ISOMYOSIN™ and ISONEINE™.

18. The method of claim 16, wherein said ISOMYOSIN™ further comprises, in combination:
Tribulus Terrestris Extract standardized to 40% Furastanol Saponins, D-RIBOSE, Magnesium Aspartate, Caffeine, Zinc Methionine, Pyridoxine, Beta-Alanine, Chasetree 4:1 Extract, Avena Savita 10:1 Extract, Epimedium 4:1, Eurycoma Longolia. Magnesium Stearate, Calcium Phosphate, Gelatin

19. The method of claim 16, said ISONEINE™ further comprises,
Glutamine, Lysine, Isoleucine, Threonine, Leucine, Methionine, Valine, Creatine Monohydrate, Trace minerals.
Magnesium Stearate, Calcium Phosphate, Gelatin

20. The method of claim 16, whereby a user procures the supplemental formulation set via electronic commerce, further comprising at least one of the Internet, and a subscription basis for order fulfillment.
